(19) United States
(12) Patent Application Publication
(10) Pub. No.: US 2024/0342008 A1
(43) Pub. Date: Oct. 17, 2024

(54) OVERLAY ADHESIVE DEVICE FOR REPAIRING AND MAXIMALLY REINFORCING LACERATIONS

(71) Applicant: Stephen Arthur Beese, Winston Salem, NC (US)

(72) Inventor: Stephen Arthur Beese, Winston Salem, NC (US)

(21) Appl. No.: 18/300,837

(22) Filed: Apr. 14, 2023

(51) Int. Cl. A61F 13/02; A61F 13/00

(52) U.S. Cl. CPC A61F 13/0233; A61F 13/00063; A61F 13/0253; A61F 13/0266; A61F 2013/00455; A61F 2013/00672

(57) ABSTRACT

A laceration closure reinforcement device includes a medical mesh impregnated with a viscous monomeric medical adhesive that completely coats a first surface and a second surface of the medical mesh. A method of reinforcing a prepared laceration includes removing the device from protective packaging; removing a first release film from the medical mesh; applying the first surface of the mesh to a prepared laceration; massaging a second release film; and removing the release film from the second surface. The mesh completely contacts all portions of the laceration, the closure strips, and a region of surrounding skin. This flexible, strong laceration repair is especially important on curvilinear surfaces where dehiscent shear and tensile forces, that occur in healing repaired minor and complex traumatic lacerations, are intensified. It increases the reliability of a laceration repair with monomeric adhesive by insuring maximum strength of the repair.

### BACKGROUND OF THE INVENTION

The present invention relates to laceration closure reinforcement and, more particularly, to an overlay adhesive device therefor.

Traumatic lacerations, wounds, and punctures are common everyday problems treated in urgent care clinics, hospital emergency departments, and primary care clinics. Treatment of lacerations is often time consuming, uncomfortable for patients, and requires considerable knowledge and skill by the clinician.

The process of laceration closure involves proper cleaning of traumatic lacerations, including debridement of non-viable tissue. Deep lacerations may require repair of subcutaneous tissues with dissolvable subcuticular sutures. Preparation may further include achieving hemostasis and employing a topical anesthetic in some cases. Under sterile conditions, lacerated skin edges are retracted together into their proper anatomic positions so that landmarks on either side (such as the vermilion border on a lip) match. Skin edges should be slightly everted when closed to promote proper healing.

The process of closing lacerated skin edges involves creating perpendicular forces to the edges to pull them into proper position. In a linear laceration, the closing forces are parallel, and most apparent when using narrow, non-elastic, reinforced adhesive closure strips, such as 3M® brand Steri-Strips®, whereby parallel closure strips are perpendicular to the skin edges.

Suture repair requires that the medical provider apply perpendicular forces to the laceration edges, but the finished repair will not display these forces. In some linear appearing lacerations that may occur on curvilinear surfaces, shear forces are present, causing the two sides of the laceration to shift, requiring closure forces at slight angles (creating counter-shear and counter-tension forces) to the skin edges to bring edges into anatomical alignment. In general, closure strips or sutures must be placed as far apart as is practical to allow closure without “gaping” of skin segments between the strips or sutures. Maintaining maximum distance between adhesive closure strips (without creating “gaping”) is especially important if a flowable adhesive is to be applied to allow maximum adhesive/skin contact.

Most lacerations encountered in primary care are traumatic, often irregular, located on curved areas and areas of high tension, and rarely linear. Lacerations may be jagged, circular, curvilinear, “flap” (when the plane of the cutting force is at a tangential angle to the skin surface), and puncture. Non-linear and curved traumatic lacerations must be managed similarly to linear injuries but require closure forces radial to skin edges (i.e., perpendicular to the tangent at a point on the laceration curve). Consequently, repair of a complex semicircular or curvilinear laceration may involve adhesive closure strips that become overlapping near the center of each curve. Thus, closure strips employed in these situations must be thin and narrow to avoid a focused buildup of closure strip material. A particularly challenging laceration is a skin “tear” frequently encountered in the elderly whose skin is often thin and fragile. These injuries are often not seen immediately after their occurrence and the ruptured epidermis/dermis may be bunched in an “accordion” fashion at one edge of the skin defect. Closure of a skin tear requires “bit by bit” traction on the skin flap edges, sequentially stretching and tacking edges with closure strips, repositioning strips as necessary, until the entire flap is properly placed. The management of such skin tears is a true art, and they are rarely amenable to the types of closure devices presently available. Currently available devices are often not suitable for lacerations on curved and irregular skin surfaces, and skin tears in elderly patients whose skin is often friable and incapable of accommodating available systems. Sutures generally work well for most traumatic lacerations but are usually inadequate for skin tears because sutures will not provide “purchase” at the skin edges, usually damaging the edges even further.

A popular and often preferred method for repairing lacerations (including surgical incisions) is the use of a flowable polymerizable monomeric skin adhesive (e.g., 2-Octyl Cyanoacrylate), applied to the entire repaired surface, that polymerizes to form a durable protective repair. The adhesive closure strips used to initially restore the damaged skin edges to their proper anatomic positions remain in place after applying the flowable adhesive. However, classic suture repair is still the predominant method for laceration repair because the irregular and challenging injuries encountered in primary care are rarely amenable to the use of available alternate laceration closure systems.

Currently available prior art laceration closure devices, generally relatively wide mesh strips, or fenestrated non-mesh strips featuring a biocompatible pressure sensitive adhesive on the skin side to allow skin traction, intended to absorb, or be covered by a flowable adhesive, are not useful for most traumatic lacerations encountered in primary care medical facilities. A variety of previous patents and patent applications describe a process of closing a laceration with a “support layer” consisting of a porous mesh, having a pressure sensitive adhesive coating on one side, allowing manual retraction of the laceration edges together followed by application of a flowable polymerizable monomeric adhesive that permeates the mesh, adhering to skin surfaces and polymerizing to form a tough protective laceration closure film. An example is US Patent Application No. 2004/010688 (Lutri et al., Jun. 3, 2004). U.S. Pat. No. 6,652,559 (Tetreault et al., Nov. 25, 2003) describes a wound/laceration closure device that consists of a flexible adhesive backed strip designed to pull together and secure the laceration edges. Employing a different approach, the strip contains discrete small perforations (0.5 mm to 3 mm) to which a polymerizable flowable adhesive is applied. Upon curing, the flowable adhesive creates discrete bonding sites between the strip and skin surfaces, without affecting the flexibility of the strip. U.S. Pat. No. 5,259,835 (Clark et al., Nov. 9, 1993) describes a typical process that also includes a sealed reservoir of liquid adhesive, packaged with a porous bonding pad, that is punctured at time of use to release liquid adhesive into the porous bonding pad prior to application to a laceration. A more recent patent, U.S. Pat. No. 11,413,370 (Jonn et al., Aug. 16, 2022) describes a flexible porous substrate with a pressure sensitive adhesive applied to one side that is employed to properly approximate wound/laceration edges, followed by application of a flowable polymerizable monomeric adhesive to the porous substrate. The adhesive polymerizes within the substrate to produce a composite wound/laceration repair.

Most prior art laceration closure devices that accommodate a flowable adhesive are adequate for linear injuries, especially relatively long surgically created incisions, but are rarely useful for commonly occurring complicated irregularly shaped traumatic lacerations. Presently available devices do not maximize laceration repair tensile and shear strength and are often not reliable in areas of high skin tension. The strength of the resulting repair is limited by the tensile and shear strengths of the adhesive film and closure strips immediately around the laceration edges. During application of a flowable adhesive, the flowing and capillary movement of the polymerizable adhesive within the material of a flexible porous substrate is limited and not consistent. Consequently, providers must actively dab, brush, roll, paint, or spread the polymerizable adhesive substance around on the surface of the flexible substrate to improve coverage and promote passive capillary distribution of the liquid adhesive through the interstices of the substrate to reach the laceration and surrounding skin surface. These devices do generally provide enhanced laceration closure force and reinforcement because of the presence of a flowable polymerizable adhesive applied to a mesh substrate. However, diffusion of monomeric adhesive may be interrupted by premature polymerization resulting in decreased contact with skin surfaces. They are especially not effective for repair of skin tears, and repair of circular/curvilinear lacerations because the devices become excessively layered at circular centers, limiting flow of adhesive into the interstices of layered material. There is no guarantee that the liquid monomer will penetrate all mesh interspaces, pressure sensitive adhesives on the mesh skin side surfaces, or the mesh/skin interface by capillary action prior to polymerization. There is also no guarantee that monomeric adhesive liquid will adequately penetrate and interact chemically with pressure sensitive adhesive applied to the mesh closure devices to solubilize the adhesive substances prior to polymerization of the monomeric laceration adhesive.

Moreover, presently available devices and methods for repairing lacerations with flowable adhesives require several coats, with time between to allow partial polymerization, creating unacceptably long procedure times. Flowable adhesives are dispensed from bottles, whose dispensing nozzles often become prematurely clogged and unusable. Furthermore, flowable topical adhesives require the healthcare provider to position the laceration/skin surface as close to horizontal as possible to minimize adhesive liquid run-off.

As can be seen, there is a need for a reliable medical mesh product with polymerizable monomeric adhesive that does not prematurely polymerize, with full penetration of the mesh, applied in a single step.

### SUMMARY OF THE INVENTION

In one aspect of the present invention, a laceration closure reinforcement device comprises a medical mesh pre-impregnated with a viscous polymerizable monomeric medical adhesive that completely coats a first surface and a second surface of the medical mesh, as well as coating all mesh fiber surfaces and filling interstitial spaces among fibers.

In another aspect of the present invention, a method of reinforcing a repaired laceration, comprises removing the laceration closure reinforcement device from a protective packaging; removing a first release film from the first surface of the medical mesh; applying the first surface of the medical mesh to a prepared laceration closed with closure strips; massaging a second release film such that the first surface of the medical mesh and polymerizable monomeric adhesive contained within completely contacts all portions of the prepared closed laceration, the closure strips, and a region of surrounding skin; and removing the second release film from the second surface.

The device described herein promotes provider efficiency by decreasing the time of laceration repair. It avoids time-consuming multiple applications of a flowable adhesive and the associated drying times of multiple adhesive applications.

It can be utilized in a broader variety of laceration repair situations, including both traumatic lacerations and surgically created incisions.

The inventive device performs as a protective cover for a repaired laceration but is not primarily a wound bandage, dressing, or covering. It is not designed to be a tool to pull laceration edges together during the repair process, to release bioactive agents or medications, or to absorb wound exudates.

The present subject matter represents an application of Fiber Reinforced Polymer (FRP) composite materials. Fiber reinforced polymers, in which a composite material made of a polymer matrix reinforced with fibers, is well known in various industries to produce high strength, high performance products. The source of the superior strength of a polymer skin adhesive reinforced with embedded mesh in this device is analogous to other fiber reinforced polymer processes such as fiberglass reinforced epoxy plastic and steel reinforced concrete. The overall properties of such composites are determined by the bonding within the fiber/polymer interface, which in turn is affected by atomic arrangement, molecular conformation, and chemical composition between fiber and matrix (polymer) components. The biomedical components listed in the present disclosure do not preclude other biocompatible materials that may result in a more effective FRP laceration reinforcing overlay.

The polymerizable monomeric viscous medical adhesive, previously applied during the manufacturing process, completely impregnates and coats all fibers and all surfaces of the laceration closure mesh prior to application to a laceration/incision. Thus, the process of relying on passive diffusion of the adhesive through mesh interstices by capillary action is avoided during the actual laceration repair process, eliminating the possibility of adhesive prematurely polymerizing within the mesh prior to reaching skin and laceration surfaces.

Additionally, the integrated reinforcing closure system with embedded mesh ensures maximum laceration closure strength by creating a composite adhered structure that spreads the repair force over a larger area than possible with sutures alone. This flexible, strong laceration repair is especially important on curvilinear surfaces where dehiscent shear and tensile forces, that occur in healing repaired minor and complex traumatic lacerations, are intensified. It increases the reliability of a laceration repair with polymerizable monomeric adhesive by insuring maximum strength of the repair.

The device disclosed herein may increase the utility of other medical adhesives such as butyl-2-cyanoacrylate which is known to have decreased laceration dehiscence strength limiting it to skin areas of low tension.

These and other features, aspects and advantages of the present invention will become better understood with reference to the following drawings, description, and claims.

### BRIEF DESCRIPTION OF THE DRAWINGS

FIG. 1 is a top perspective view of a laceration repair and reinforcing overlay device that reinforces lacerations closed by an adhesive according to an embodiment of the present invention;

FIG. 2 is a side elevation view thereof;

FIG. 3 is a top perspective view thereof, shown in a partially activated mode; and

FIG. 4 is a perspective view showing a process according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a single step, time efficient process for reinforcing a laceration with a fiber reinforced pre-impregnated viscous monomeric medical adhesive overlay—a mesh article whose fibers are pre-impregnated and surrounded on all surfaces with polymerizable monomeric adhesive, protected by release films, and pre-packaged in hermetically sealed packaging to prevent premature polymerization of the monomeric adhesive.

In a single step, the device (after removal of release films) is applied directly to the prepared laceration, ensuring complete immediate contact with all areas of the closed laceration and surrounding skin. The adhesive immediately begins to polymerize to effect laceration closure.

The use of this device follows initial manual restoration of laceration edges to their proper anatomic position with appropriately arranged narrow, pressure adhesive-coated adhesive closure strips. Numerous generic and proprietary closure strips are currently available. The viscous polymerizable monomeric adhesive is completely sequestered within the fiber mesh component. Readily polymerizable topical (viscous) monomer adhesives well known in the art include the α-cyanoacrylates. The most effective, safe, and commonly used adhesive in this class is 2-octyl cyanoacrylate which has a breaking strength close to that of a 5-0 monofilament suture. However, any biocompatible polymerizable monomeric medical adhesive known in the art may be used.

According to an embodiment of the present invention, additional components in the viscous polymerizable monomer adhesive formulation (e.g., cyanoacrylate) may include but are not limited to free-radical stabilizers, anionic stabilizers, plasticizers, thickeners etc. The details are described in U.S. Pat. Nos. 5,981,621 and 6,433,096, the contents which are incorporated by reference herein in their entireties. To prevent premature polymerization of adhesive contained within and around the mesh while stored in packaging, the adhesive compound may contain stabilizers known in the art for a given class of adhesive. The adhesive may contain a plasticizer, known in the art, to produce a more pliable and tissue-compatible product that flexes with the skin and minimize stiffness that will be introduced by the presence of an incorporated mesh. The adhesive may contain an appropriate thickener, known in the art, to balance a viscosity to enable penetration of mesh openings while preventing outflow. The adhesive compound may further include a catalyst, known in the art, to facilitate the acceleration of polymerization. However, monomeric cyanoacrylate adhesive generally begins to polymerize in the presence of moisture upon contacting a laceration/skin surface.

The reinforcing mesh material is not particularly limited, provided it is flexible but not elastic or distensible. Reinforcing mesh may be formed of either synthetic or natural materials. The mesh material may be either woven or non-woven. The flexible material may be, for example, any suitable polymeric film, a woven fabric, knitted fabric, a non-woven fabric, mixture thereof, or the like known in the art as compatible with cyanoacrylate adhesives.

Mesh size could be about 8 to 18 openings per inch, sized to maximize capture of the adhesive within mesh openings, yet minimize stiffness of the polymerized assembly which could result from decreased size mesh openings and increased density of mesh fibers. The mesh/adhesive overlay is sized to completely cover the laceration and extend adequately beyond its borders to maximize the reinforcing feature of the mesh. Medical mesh may be cut in several different sizes (e.g., one inch by 2-inch, 2-inch by 4-inch, etc.) to cover a variety of commonly encountered lacerations and allow options for maximally reinforcing larger lacerations.

Four release films may be provided to protect the mesh/monomer assembly, two on each side, each coated with silicon (or other adhesive [e.g., cyanoacrylate] incompatible coating to which the adhesive [e.g., cyanoacrylate] will not adhere, known in the art).

Air and water impermeable packaging (i.e., hermetically sealed), stable in the presence of medical repair adhesive, may be used to protect and store the mesh/adhesive assembly prior to usage, preventing premature polymerization of the monomeric adhesive.

The viscous monomeric adhesive contained within and surrounding the reinforcing mesh polymerizes to form a flexible unitized repair whose closure strength is relatively proportional to the skin area covered. The mesh adhesive device ensures maximum adhesive contact and maximum closure strength by spreading closure force over a definable area.

In some embodiments, a multiple overlay system according to an embodiment of the present invention provides increased closure strength in body skin areas expected to have increased tensile/shear forces that could result in dehiscence of repaired laceration edges, without layered build-up of the ends of mesh strip closure devices at the centers of circular lacerations and skin tears. An overlay of multiple mesh/adhesive complexes extending well beyond the laceration edges provides greater strength to resist increased laceration dehiscence forces. The embedded fibers absorb the tensile and shear forces created at the laceration edges during movement/flexion of surrounding skin. Thus, simply increasing the area of skin coverage around a repaired laceration increases the strength of the repair. The larger the area covered by a mesh/polymer repair film, the greater the strength of the repair and its ability to prevent laceration dehiscence.

The composite overlay disclosed herein may be manufactured by applying cyanoacrylate or other polymerizable monomeric adhesives known in the art to mesh devices by appropriate manufacturing methods. The mesh is saturated and reliably and completely coated on all surfaces with viscous polymerizable monomeric adhesive during the manufacturing process and may be “sandwiched” between two release films. The entire assembly may be sealed within protective packaging.

In another embodiment, the release films may be sealed to serve as the hermetically sealed packaging for the device. Manufacture of the device may be outsourced to a commercial or specialized medical product enterprise.

In a method of using the inventive overlays, after preparation and cleansing of a traumatic laceration or surgical skin incision as well known in the art, the edges are first restored to their proper anatomic position with appropriately arranged narrow adhesive closure strips. Numerous generic and proprietary strips are currently available. An effective product is the 3M@ Steri-Strip® Reinforced Skin Closure containing non-elastic reinforcing polymer filaments and an effective pressure sensitive acrylate adhesive. For example, a convenient size may be 3 mm wide by 3 inches long. In some situations, it may be appropriate to cut the strip into 1.5 mm wide strips can for small lacerations. An appropriately sized polymerizable monomeric adhesive pre-impregnated mesh assembly may then be selected and removed from its protective packaging. Once a release paper is removed, the user may apply the exposed adhesive surface directly over the closed laceration and previously applied closure strips. After applying the mesh overlay (pre-impregnated with polymerizable monomeric adhesive), the overlay intimately adheres to the laceration edges, previously applied closure strips, and surrounding areas of intact skin. The remaining release film may be gently massaged to ensure complete contact with the laceration, previously applied closure strips, and surrounding skin. The remaining release film is then peeled off. Finally, the applied mesh assembly is inspected to ensure complete contact with the repaired laceration, closure strips (which become an integral component of the reinforcing complex) and surrounding skin and may be gently massaged to conform to the skin contours and to ensure maximum adherence to the laceration. The viscous polymerizable monomeric medical adhesive immediately, intimately, and maximally contacts all surfaces of the laceration edges, closure strips, and surrounding skin. Polymerization of the monomeric adhesive quickly initiates, catalyzed by the presence of moisture on laceration/skin surfaces. The process thus requires only a one-step application of topical medical adhesive. For long lacerations, additional adhesive assemblies may be placed adjacent to each other, slightly overlapping. For areas of high skin tension, the width of the device (perpendicular to the average axis of the laceration) applied may be increased.

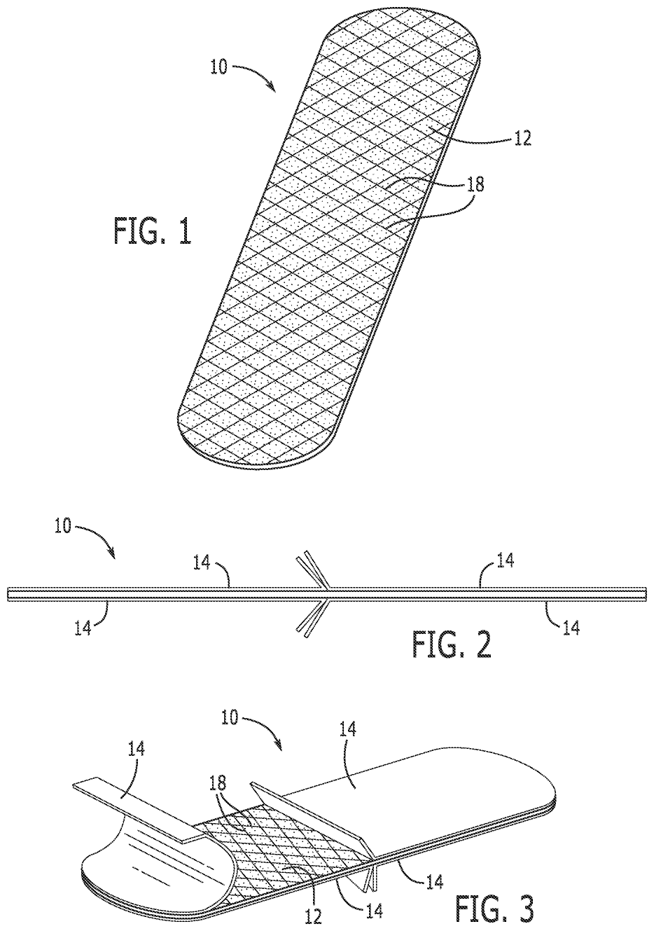

Referring to FIGS. 1 through 4, FIG. 1 illustrates an overlay device 10 for maximally reinforcing a laceration, comprising a fiber medical reinforcing mesh 18 pre-impregnated (and thereby completely coated) with a viscous polymerizable monomeric medical adhesive 12.

FIG. 2 is a schematic illustration of the device 10 sandwiched between four release films 14 (two on each side). The release films 14 are sized larger than the mesh/adhesive complex 10 to allow finger grasping areas. In another possible release film embodiment (not shown in drawings), each side of the mesh/adhesive complex could be protected by a single release film, each film longer in length and width than the mesh/adhesive complex to provide edge grasping areas.

FIG. 3 is a schematic illustration showing removal of one release film 14, after removal from the protective package (not shown), and showing the exposed reinforcing mesh 18, completely impregnated and coated with the viscous polymerizable monomeric adhesive 12, while the device 10 is protected by its remaining two release films 14.

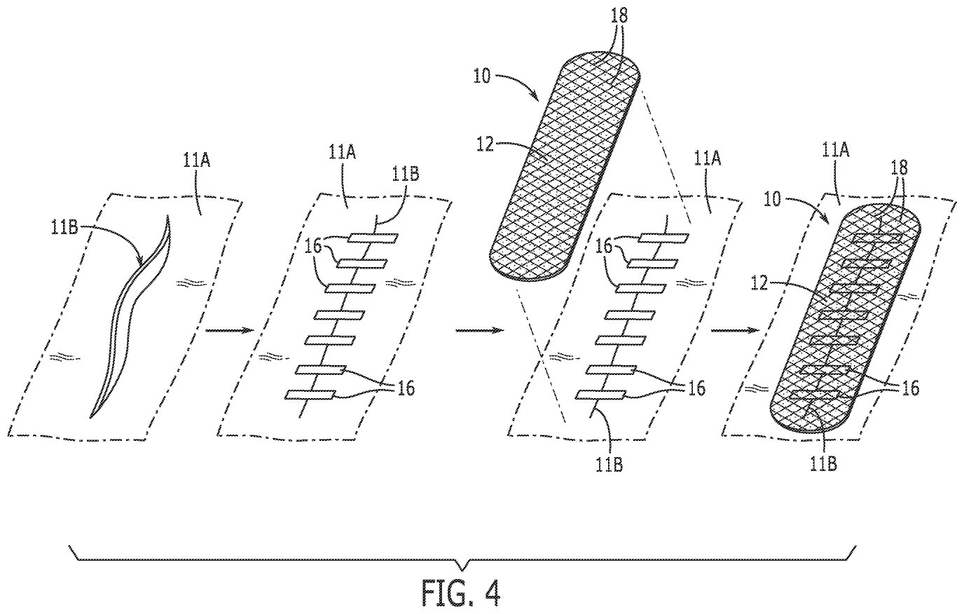

FIG. 4 is a schematic illustration of the process of applying the device 10 to a prepared closed laceration 11B. After standard preparation and cleansing of the skin 11A defect, the wound edges are restored to their proper anatomic position with appropriately arranged, narrow, non-elastic adhesive closure strips 16. The closure strips 16 must be of sufficient length to allow skin 11A purchase beyond the laceration edges 11B to allow adequate retraction of laceration edges 11B.

It should be understood, of course, that the foregoing relates to exemplary embodiments of the invention and that modifications may be made without departing from the spirit and scope of the invention as set forth in the following claims.

What is claimed is:

1. A laceration closure reinforcement device comprising: a medical mesh impregnated with a viscous monomeric medical adhesive that completely coats a first surface and a second surface of the medical mesh.

2. The laceration closure reinforcement device of claim 1, further comprising a hermetically sealed and sterile package enclosing the medical mesh.

3. The laceration closure reinforcement device of claim 1, wherein the viscous monomeric medical adhesive comprises an a-cyanoacrylate.

4. The laceration closure reinforcement device of claim 1, wherein the viscous monomeric medical adhesive further comprises at least one adjuvant selected from the group consisting of: a free-radical stabilizer, an anionic stabilizer, a plasticizer, a thickener, a catalyst, and any combination thereof.

5. The laceration closure reinforcement device of claim 1, further comprising a release film removably adhered to each surface of the medical mesh.

6. The laceration closure reinforcement device of claim 5, wherein the protective release films are hermetically sealed together, enclosing the medical mesh.

7. A method of reinforcing a repaired laceration, comprising:
removing the laceration closure reinforcement device of claim 1 from a protective packaging;
removing a first release film from the first surface of the medical mesh;
applying the first surface of the medical mesh to a prepared laceration closed with closure strips;
massaging a second release film such that the first surface of the medical mesh completely contacts all portions of the prepared closed laceration, the closure strips, and a region of surrounding skin; and
removing the second release film from the second surface.

8. The method of claim 7, wherein the step of applying comprises iteratively applying additional laceration closure reinforcement devices in an adjacent configuration, with edges thereof overlapping, such that laceration closure reinforcement devices are applied to cover the prepared closed laceration and the region of surrounding skin.
